(19) United States
(12) Patent Application Publication
(10) Pub. No.: US 2006/0141595 A1
(43) Pub. Date: Jun. 29, 2006

(54) Process for continuous manufacture of invert sugar syrup and alcohol

(75) Inventors: Asmita Ashutosh Prabhune, Maharashtra (IN); Sanjeevani Amrit Pardhy, Maharashtra (IN); Shilpa Shirish Deshpande, Maharashtra (IN); Archana Vishnu Pundle, Maharashtra (IN); Sulabha Kishore Karandikar, Maharashtra (IN)

Correspondence Address: NIXON & VANDERHYE, PC, 901 NORTH GLEBE ROAD, 11TH FLOOR, ARLINGTON, VA, 22203, US

(73) Assignee: COUNCIL OF SCIENTIFIC AND INDUSTRIAL RESEARCH, New Delhi (IN)

(21) Appl. No.: 11/024,039

(22) Filed: Dec. 29, 2004

(51) Int. Cl. C12P 7/06; C12P 19/02

(52) U.S. Cl. 435/161; 435/105

(57) ABSTRACT

The present invention relates to an improved process for the manufacture of invert sugar syrup and alcohol capable of operating in the continuous mode for enzymatic hydrolysis of sucrose or sucrose containing sources for the production of invert syrup and thereby alcohol fermentation and using a recyclable solid catalyst

### FIELD OF THE INVENTION

The present invention relates to an improved process for the continuous manufacture of invert sugar syrup and alcohol The present invention particularly relates to a process capable of operating in the continuous mode for enzymatic hydrolysis of sucrose or sucrose containing sources for the production of invert syrup and thereby alcohol fermentation. Still more particularly it relates to the said process using a solid catalyst which can be recycled, reused over a long period in continuous as well as in batch mode.

### BACKGROUND OF THE INVENTION

The invert sugar syrups (an equimolar mixture of glucose and fructose) have wide applications in the food and pharmaceutical industries, because of it's functionally more desirable properties i.e. high osmotic pressure, high solubility and humid nature. This enzyme catalyses the conversion of sucrose into glucose and fructose in equimolar concentrations Their use in confectionery ensures that the product remains fresh and soft even when kept for a long time They are also used in the production of non-crystallizing ice creams, condensed milk, infant foods, jams, and substitute for honey and in the production of sugar syrup in pharmaceutical industry. It has a potential use in the production of alcoholic beverages, lactic acid, and glycerol. Other important applications of Invert sugars are

1) Intravenous injectables for treatment of certain pathological conditions such as diabetes.

2) In paper and tobacco industries because of its humectency.

3) As OmegaZyme caplets used as a digestive health product.

The conventional method for manufacturing invert sugar syrup involves an acid hydrolysis of sucrose Enzymatic hydrolysis of sucrose to invert sugar is preferred to acid hydrolysis, as it does not result in the production of furfural, oligosaccharides and other undesirable flavors.

The conventional method for manufacturing invert sugar syrup involves an acid hydrolysis of sucrose. However, such crude acid hydrolysis has a low conversion efficiency, high energy consumption and thus high cost of production. The acid hydrolyzed product also contains impurities introduced by uncontrollable parameters during inversion

There is a long history for enzyme (protein) immobilization using solid supports via adsorption, encapsulation and covalent linking. One of the most widely used methods for immobilizing enzymes is encapsulation inside sol-gel silica. However, due to small pore size and non-open pore structure, most studies showed lower specific activity than that of the free enzyme in solution. Unlike sol-gel silica, mesoporous silica provides a rigid, uniform open-pore structure Functionalized mesoporous silica has exhibited a very high affinity for binding heavy metal ions with mercapto functional groups. Functionalized mesoporous silica extrudates would have great potential for high enzyme loading, provided that 1) the pore size is sufficiently large for the enzyme to be “comfortably” hosted and also for its substrate and product to access and diffuse easily through open pore channels and 2) appropriate functional groups provide high affinity for protein molecules. Recently, mesoporous silica has begun to attract attention for enzyme immobilization

### OBJECTS OF THE INVENTION

The main object of the invention is to produce functionalized mesoporous silica extrudates which are easy to handle and can be removed during downstream processing.

It is another object of the invention to immobilize invertase on such extrudates

Another object of the invention is to immobilize the enzyme on the extrudates using a crosslinking agent such as glutaraldehyde to improve the shelf life.

A further object of the invention is to grow yeast cells in a nutrient medium having a carbon and nitrogen source, and to use these cells for production of alcohol using immobilized enzyme.

### SUMMARY OF THE INVENTION

Accordingly, the present invention provides a process for the continuous manufacture of invert sugar syrup and alcohol, the process comprising:
(a) immobilizing an invertase enzyme on a functionalized mesoporous silica powder or extrudate by mixing the invertase enzyme with functionalized mesoporous silica in the presence of a crosslinking agent to obtain an immobilized and cross-linked invertase complex,
(b) separating the immobilized and cross-linked complex;
(c) subjecting a sucrose source to fermentation in the presence of the complex in a fermentation medium using yeast species to obtain an invert syrup in broth;
(d) separating the yeast cells to obtain the invert syrup and unutilized sucrose as a mixture,
(e) subjecting a mixture of invert syrup and unutilized sucrose, and fresh sucrose solution to fermentation using yeast cells to obtain alcohol.

In one embodiment of the invention, the invertase enzyme is β fructofuranosidase

In another embodiment of the invention, the cross linking agent is a bifunctional agent comprising glutaraldehyde.

In yet another embodiment of the invention, step (a) is carried out under constant agitation at a temperature in the range of 10-20° C.

In another embodiment of the invention, in step (c) the yeast species is selected from the group consisting of Kluyveromyces marxianus (NCYC 2675) and Saccharomyces cerevisiae (NCIM 3049).

In another embodiment of the invention, in step (c) the sucrose source is fermented for a period of 16 hrs to 48 hrs at a temperature of 30° C. to 50° C. under aerobic condition.

In another embodiment of the invention, the functionalized mesoporous silica is prepared from mesoporous silica by treating mesoporous silica with a functionalizing agent having an amino group such as aminopropyl trimethoxy silane

In another embodiment of the invention, the functionalization of mesoporous silica is effected before mixing with Boehemite.

In another embodiment of the invention, the functionalization of mesoporous silica is effected after mixing with Boehemite.

In yet another embodiment of the invention, the mesoporous silica has a pore size in the range of 40 to 90 Å

### DETAILED DESCRIPTION OF THE INVENTION

The present invention provides for the use of mesoporous silica with large pores (40-90 Å) that are sequentially functionalized to thereby yield high protein loading and enhanced enzyme activity. Mesoporous SBA-15 has potential application as support for enzyme immobilization due of its high surface area and high pore volume. Functionalization with organosilane to generate a monolayer of charged groups on surface facilitates uniform distribution of the enzyme molecules in the channels of the mesoporous silica. Enzyme treated mesoporous silica gives additional advantage after cross linking. This results in a stable, highly active, recyclable and long life solid catalyst. The process of the present invention also provides for acceleration of rate of alcohol production using immobilized invertase on functionalized silica. Addition of plain silicalite showed no increase in alcohol production throughout the fermentation

The present invention attempts to produce functionalized mesoporous silica extrudates, which are easy to handle and can be removed during downstream processing. Invertase enzyme is immobilized on the extrudates using a crosslinking agent such as glutaraldehyde to improve the shelf life. The yeast cells are grown in a nutrient medium having a carbon and nitrogen source, and used for production of alcohol using immobilized enzyme.

The process of the invention results in continuous manufacture of invert sugar syrup and alcohol. The process comprises immobilizing a commercially available invertase enzyme such as β fructofuranosidase on a functionalized mesoporous silica powder or extrudate by mixing the invertase enzyme with functionalized mesoporous silica in the presence of a crosslinking agent under constant agitation at a temperature in the range of 10-20° C. This results in formation of a immobilized invertase complex. This immobilized and cross linked complex is separated by conventional methods. A sucrose source is subjected to fermentation in the presence of the complex in a conventional fermentation medium using yeast species such as Kluyveromyces marxianus (NCYC 2675) or Saccharomyces cerevisiae (NCIM 3049) for a period of 16 hrs to 48 hrs at a temperature of 30° C. to 50° C. under aerobic condition to obtain the invert syrup in broth. The yeast cells are separated to obtain the invert syrup along with unutilized sucrose. This mixture of the invert syrup and unutilized sucrose and fresh sucrose solution is subjected to fermentation using yeast cells to obtain alcohol.

The invertase enzyme can be any commercially available invertase such as β fructofuranosidase. Functionalized mesoporous silica is prepared from mesoporous silica by treating mesoporous silica with a functionalizing agent having an amino group such as aminopropyl trimethoxy silane by a known method. The crosslinking agent is a bifunctional reagent such as glutaraldehyde.

In a feature of the present invention the functionalization of mesoporous silica was done before/after mixing with Boehemite

### EXAMPLE 1

This example illustrates the preparation of extrudates of functionalized mesoporous silica. The binder used for making the extrudates is Boehemite. 1 g of pre-functionalised mesoporous silica is mixed with 0.3 g Boehemite. This is then soaked with water to make extrudates of 1 mm diameter and 1.4 mm length. The extrudates thus made were dried in an oven at 100° C. and, used for immobilization.

### EXAMPLE 2

Extrudates B (Method of Preparation of These Extrudates is Similar to Example 1)

Mixing of Boehemite with mesoporous silica was done before functionalization of silica.

### EXAMPLE 3

This example illustrates preparation of following complexes (silica+invertase) using mesoporous silica extrudates A/extrudates B

Complex A: Extrudates A−0.25 g.+2.5 ml of 0.05M acetate buffer (pH 4.5)+0 25 ml. enzyme

Complex B. Extrudates B−0.25 g.+2.5 ml of 0.05 M acetate buffer+0.25 ml enzyme.

Complex C Extrudates A−0.25 g.+25 ml of 0.05 M acetate buffer+0.25 ml enzyme+50 μl glutaraldehyde.

Complex D. Extrudates B−0.25 g.+2 5 ml of 0.05M acetate buffer+0 25 ml enzyme+50 μl glutaraldehyde

Enzyme activity of supernatants and complexes were checked by invertase assay as described by Gascon and Lampen (JBC 1968, vol. 243, p 1573-1577) and reducing sugar determined as described by Miller (Analytical Chem 1959, vol 3 p426)

TABLE 1
Efficiency of complexes

| Complex | % efficiency as compare with free enzyme |
|---|---|
| A | 62% |
| B | 70% |
| C | 79% |
| D | 85% |

### EXAMPLE 4

As seen in Example 3, glutaraldehyde containing complexes (complex C and complex D in table 1) show higher invertase activity than complex without glutaraldehyde (complex A and complex B in table 1). Therefore four types of complexes were further prepared using glutaraldehyde as crosslinking agent.

Complex I The mixture consists of functionalized mesoporous silica extrudate A (0.15 g)+1 ml acetate buffer (0.05M)+25 μl glutaraldehyde. This was allowed to stand for 15 minutes (with intermittent stirring) at room temperature followed by decantation to remove unbound glutaraldehyde. To the residue 100 μl of the enzyme and 1 ml of 0.05M acetate buffer was added. Enzyme activity of the complex was determined.

Complex II. Same procedure was repeated as for Complex I except that extrudate B was used

Complex III: Functionalized mesoporous silica, extrudatesA+1 ml acetate buffer (0.05M) and 100 μl enzyme were mixed and allowed to stand for 15 minutes (with intermittent stirring) at room temperature. The supernatant was decanted to remove any free enzyme followed by addition of 25 μl glutaraldehyde and 1 ml of (0.05M) acetate buffer. Enzyme activity of the complex was determined.

Complex IV: Procedure is same as complex III except that extrudate B was used.

TABLE 2
% efficiency as compared to free enzyme.

| Complex | % Efficiency |
|---|---|
| I | 75% |
| II | 70% |
| III | 60% |
| IV | 55% |

### EXAMPLE 5

All four complexes in Example 3 and Example 4 were given repeated washings with 0 05M acetate buffer. Activity of the complexes and the supernatants were checked after each washing Activities of the complexes were observed to be constant after repeated washings

### EXAMPLE 6

Cells of Saccharomyces cerevisiae NCIM 3049 were grown in 5% MSYP with following composition Malt extract 3 g, yeast extract 3 g, peptone 5 g; sucrose 50 g at 30° C. for 48 hours on shaker. Cells were allowed to settle for three days. Supernatant was discarded and sedimented cells used for fermentation.

### EXAMPLE 7

50 ml of 5 g %, 10 g %, 15 g %, 20 g % and 25 g % sterile sucrose solutions were prepared in 250 ml flasks in 3 sets. 1 ml of prepared yeast cells approximately 1 gm were added to each flask Complex A at concentration—0.5 gm was added to second set of flask (Table 4) and Complex C at concentration of 0.5 gm was added to third set of flasks (Table 5). The flasks were kept at 30° C. on the shaker. 7 ml of broth was removed aseptically from each flask at intervals of 3 hrs, 6 hrs, 9 hrs, 24 hrs and 48 hrs, from each set and each sucrose concentration.

### EXAMPLE 8

5 ml of the removed broth was mixed with 4 ml of distilled water in round bottom flask and distilled at 100° C. 5 ml of distillate was collected, and alcohol estimated at 486 nm using cerric ammonium nitrate method. (Analyst 1952, 77 p325-497.)

TABLE 3
Ethanol % (w/v) produced without addition of complex
Time in hours

| | | | | | |
|---|---|---|---|---|---|
| Concentration | | | | | |
| of sucrose | 3 hrs | 6 hrs | 9 hrs | 24 hrs | 48 hrs |

| | | | | | | |
|---|---|---|---|---|---|---|
| 5 | g % | 1.56 | 2.25 | 2.42 | 2.31 | 2.3 |
| 10 | g % | 3.12 | 4.29 | 4.76 | 4.88 | 4.9 |
| 15 | g % | 3.26 | 4.88 | 5.21 | 6.77 | 7.39 |
| 20 | g % | 3.66 | 4.92 | 5.5 | 7.16 | 7.23 |
| 25 | g % | 4.12 | 4.96 | 5.6 | 7.29 | 7.59 |

TABLE 4
Ethanol % (w/v) produced with addition of complex A
Time in hours

| | | | | | |
|---|---|---|---|---|---|
| Concentration | | | | | |
| of sucrose | 3 hrs | 6 hrs | 9 hrs | 24 hrs | 48 hrs |

| | | | | | | |
|---|---|---|---|---|---|---|
| 5 | g % | 1.81 | 2.41 | 2.48 | 2.46 | 2.43 |
| 10 | g % | 3.23 | 4.36 | 4.86 | 4.96 | 4.92 |
| 15 | g % | 3.8 | 4.91 | 5.72 | 7.46 | 7.41 |
| 20 | g % | 3.87 | 5.12 | 5.72 | 7.89 | 7.91 |
| 25 | g % | 5.46 | 5.53 | 6.17 | 7.92 | 7.95 |

TABLE 5
Ethanol % (w/v) produced with addition of complex C

| | | | | | |
|---|---|---|---|---|---|
| Concentration | | | | | |
| of sucrose | 3 hrs | 6 hrs | 9 hrs | 24 hrs | 48 hrs |

| | | | | | | |
|---|---|---|---|---|---|---|
| 5 | g % | 1.83 | 2.47 | 2.42 | 2.31 | 2.22 |
| 10 | g % | 3.33 | 4.46 | 4.92 | 4.96 | 4.8 |
| 15 | g % | 3.95 | 5.2 | 5.88 | 7.8 | 7.3 |
| 20 | g % | 3.87 | 5.22 | 5.80 | 5.11 | 7.91 |
| 25 | g % | 5.5 | 5.53 | 6.2 | 8.34 | 8.8 |

### ADVANTAGE OF PRESENT INVENTION

Present invention increases the rate of alcohol fermentation because the complex sugar is converted into simpler sugars by immobilized invertase in addition to yeast species used for fermentation. This increases the intake of sugar and hence the faster fermentation. This is the first report where enzyme has been immobilized in functionalized mesoporous silica and extrudates, which facilitates easy handling and reusability. Crosslinking of this enzyme on the matrix with bi functional reagent increases the shelf life and reduces leaching of the enzyme

We claim:

1. A process for the manufacture of invert sugar syrup and alcohol, the process comprising:
(a) immobilizing an invertase enzyme on a functionalized mesoporous silica powder or extrudate by mixing the invertase enzyme with functionalized mesoporous silica in the presence of a crosslinking agent to obtain an immobilized and cross-linked invertase complex;
(b) separating the immobilized and cross-linked complex;
(c) subjecting a sucrose source to fermentation in the presence of the complex in a fermentation medium using yeast species to obtain an invert syrup in broth;
(d) separating yeast cells to obtain the invert syrup and unutilized sucrose as a mixture,
(e) subjecting a mixture of invert syrup and unutilized sucrose, and fresh sucrose solution to fermentation using yeast cells to obtain alcohol

2. A process as claimed in claim 1 wherein the invertase enzyme is β fructofuranosidase.

3. A process as claimed in claim 1 wherein the cross-linking agent is a bifunctional agent comprising glutaraldehyde.

4. A process as claimed in claim 1 wherein step (a) is carried out under constant agitation at a temperature in the range of 10-20° C.

5. A process as claimed in claim 1 wherein in step (c) the yeast species is selected from the group consisting of Kluyveromyces marxianus (NCYC 2675) and Saccharomyces cerevisiae (NCIM 3049).

6. A process as claimed in claim 1 wherein in step (c) the sucrose source is fermented for a period of 16 hrs to 48 hrs at a temperature of 30° C. to 50° C. under aerobic condition.

7. A process as claimed in claim 1 wherein the functionalized mesoporous silica is prepared from mesoporous silica by treating with a functionalizing agent having an amino group

8. A process as claimed in claim 7 wherein the functionalising agent is aminopropyl trimethoxy silane.

9. A process as claimed in claim 7 wherein functionalization of mesoporous silica is effected before mixing with Boehemite

10. A process as claimed in claim 7 wherein functionalization of mesoporous silica is effected after mixing with Boehemite.

11. A process as claimed in claim 1 wherein the mesoporous silica has a pore size in the range of 40 to 90 Å

12. A process as claimed in claim 1 which is continuous or batch process

13. A process as claimed in claim 1 wherein the catalyst is recycled
